(19) United States
(12) Patent Application Publication
(10) Pub. No.: US 2026/0294421 A1
(43) Pub. Date: Oct. 1, 2026

(54) RACHET-BASED RETRACTOR AND DISTRACTOR APPARATUS

(71) Applicant: ATLAS SPINE, INC., Jupiter, FL (US)

(72) Inventor: Matthew G. Baynham, Jupiter, FL (US)

(21) Appl. No.: 19/688,661

(22) Filed: May 27, 2026

Related U.S. Application Data
(63) Continuation-in-part of application No. 18/962,484, filed on Nov. 27, 2024, now Pat. No. 12,661,100.
Application No. 18/962,484 is a continuation of application No. 17/492,353, filed on Oct. 1, 2021, now Pat. No. 12,185,987.
(60) Provisional application No. 63/086,822, filed on Oct. 2, 2020.

(51) Int. Cl. A61B 17/02; A61B 17/70; A61B 17/68

(52) U.S. Cl. CPC A61B 17/0206; A61B 17/02; A61B 17/0218; A61B 17/7035; A61B 17/7053; A61B 17/7076; A61B 17/025; A61B 2017/0256; A61B 2017/681

(57) ABSTRACT

A rachet-based retractor and distractor apparatus for use in surgical procedures includes a first member and a second member coupled at an offset pivot interface. A ratchet member with a ratchet tooth drive is supported by the first member and cooperates with a ratchet lever and adjustment screw on the second member to control spacing between opposing arms. Each arm supports a blade configured to engage a surgical site, with wires forming expandable hoops for securing the blades to bone anchors. A housing at the pivot supports a medial retractor arm that is advanced and retracted by a screw-driven rack and pinion mechanism. The apparatus further includes adjustable blade features, including splined attachment pins, extension portions, and removable blade brackets. Together, these components allow controlled adjustment of blade position and access to the surgical site during a procedure.

### PRIORITY CLAIM

In accordance with 37 C.F.R. § 1.76, a claim of priority is included in an Application Data Sheet filed concurrently herewith. The instant invention is a continuation in part (CIP) and claims priority to U.S. patent application serial number 18/962,484, entitled “BONE ANCHOR BASED RETRACTOR/DISTRACTOR” filed November 27, 2024, which claims priority to U.S. Patent Application No. 17/492,353 entitled “BONE ANCHOR BASED RETRACTOR/DISTRACTOR”, filed October 1, 2021 having registration number 12,185,987, Issued on January 7, 2025, which claims priority to U.S. Provisional Patent Application No. 63/086,822 entitled “BONE ANCHOR BASED RETRACTOR/DISTRACTOR”, filed October 2, 2020; the contents of the above referenced application are incorporated herein by reference in their entirety.

### FIELD OF THE INVENTION

This invention is related to the field of spinal surgery and, more specifically, to a simultaneous tissue retraction and disc space distraction device.

### BACKGROUND OF THE INVENTION

Surgical approach for addressing spinal injuries and pathologies commonly involves implanting various devices for correcting the spine, including intervertebral stabilization, distraction, decompression, joint fusion, or combinations thereof. Bone screws are most commonly used for anchoring of plates, rods, and tethers used in various combinations to achieve desired correction to the spine. Conventionally, there are three modes of access to the spine for achieving delivery of spinal correction devices. These general modes include anterior (through the abdominal cavity), posterior (including transforaminal), and lateral. Modes of access for achieving fusion between lumbar vertebrae: anterior access known as "ALIF" (Anterior Lumbar Interbody Fusion); posterior access known as "PLIF" (Posterior Lumbar Interbody Fusion); and minimally invasive posterior access known as "TLIF".

It is preferable, when operating on the spine, to employ the least invasive surgical approach possible for achieving correction to minimize trauma, associated pain and blood loss experienced by the patient, to improve recovery time and outcomes, and to reduce operating room time and costs. In all modes of approach, one or more special retractors and tubes are typically used to dissect and displace tissue and expose the vertebrae, and other instruments are used to release the annulus.

There is a need for a surgical approach and associated instrumentation and devices that avoid the existing complications known in the art with the various modes of spinal access. More specifically, there is a need for advances with instrumentation and surgical technique to allow for the more desirable prone patient positioning during spinal access surgery, combined with the benefits of lateral access to the target intervertebral space.

### SUMMARY OF THE INVENTION

Disclosed is a retractor/distractor apparatus for use in spinal procedures. The apparatus includes a first member and a second member that are joined at an offset pivot point. The first member has an upper leg that carries a ratchet member and a lower leg that connects to a first arm. The second member has an upper end that carries a ratchet adjustment mechanism and a lower end that connects to a second arm. The ratchet member includes a ratchet tooth drive that cooperates with a ratchet lever on the second member. Rotation of a ratchet adjustment screw moves the second member relative to the first member along the ratchet member. This movement changes the spacing between the first and second arms to control retraction at the surgical site.

The first and second arms support respective blades that engage the surgical site. Each blade is coupled to its arm by an attachment pin. Each blade also includes an internal pathway that guides a wire. The wire forms a loop at a lower end of the blade. The loop can be expanded or contracted to secure the blade to a bone anchor. The blades can be attached to the bone anchors before the retractor/distractor apparatus is installed. This arrangement allows the surgeon to place the blades first and then connect the apparatus afterward during operation.

A housing is positioned at the pivot point and supports a medial retractor arm. The housing includes a medial actuation screw that engages a rack member. Rotation of the medial actuation screw drives the rack member and moves the medial retractor arm forward and backward. In certain embodiments, the rack member includes inner teeth that engage a pinion supported by the housing. A medial locking actuator may be provided to hold the medial retractor arm in position. The actuator can be engaged to prevent movement in one direction and released to permit movement in the opposite direction.

The ratchet member may include a series of outer locking teeth. These teeth permit movement of the second member in one direction and resist movement in the opposite direction. The ratchet lever includes an end that engages the locking teeth. A click spring biases the lever into engagement with the teeth. When the ratchet lever is pressed, the click spring compresses and the lever disengages from the teeth. This allows the second member to move in the opposite direction and reduces the applied retraction force.

In certain embodiments, the arms are coupled to the members by worm gears. Rotation of the worm gears adjusts the angular orientation of the blades. The attachment pins may include splined surfaces that engage corresponding splined openings for more angular control of the blades. The blades may be mounted to blade brackets that include catch posts received within apertures of the arms. Release buttons can be used to remove the blade assemblies entirely. A detent may also be provided for the medial retractor arm to hold the medial blade in place. The detent includes a spring-biased protrusion that engages recesses on a medial blade to hold the blade in selected positions.

The retractor/distractor apparatus gives the surgeon control over how the surgical site is opened, with independent adjustment of the medial retractor and the positioning of each blade. It securely engages bone anchors and can be adjusted in a steady and predictable way during the procedure.

An objective of the present invention is to provide a retractor/distractor apparatus that allows controlled expansion of a surgical site using a ratchet-based mechanism. The spacing between opposing arms can be adjusted incrementally, which helps maintain stable and controlled tissue retraction during a procedure.

Another objective of the present invention is to provide a retractor/distractor apparatus with an independently adjustable medial retractor. The medial retractor can be advanced or withdrawn relative to the surgical site to improve visibility and access without disturbing the position of the primary retraction arms.

Yet another objective of the present invention is to provide a modular blade system that can be secured to bone anchors before the retractor/distractor apparatus is attached. This allows the blades to be positioned first and then connected to the apparatus, which can make placement easier during the procedure.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a perspective view of a retractor/distractor apparatus in a closed position;

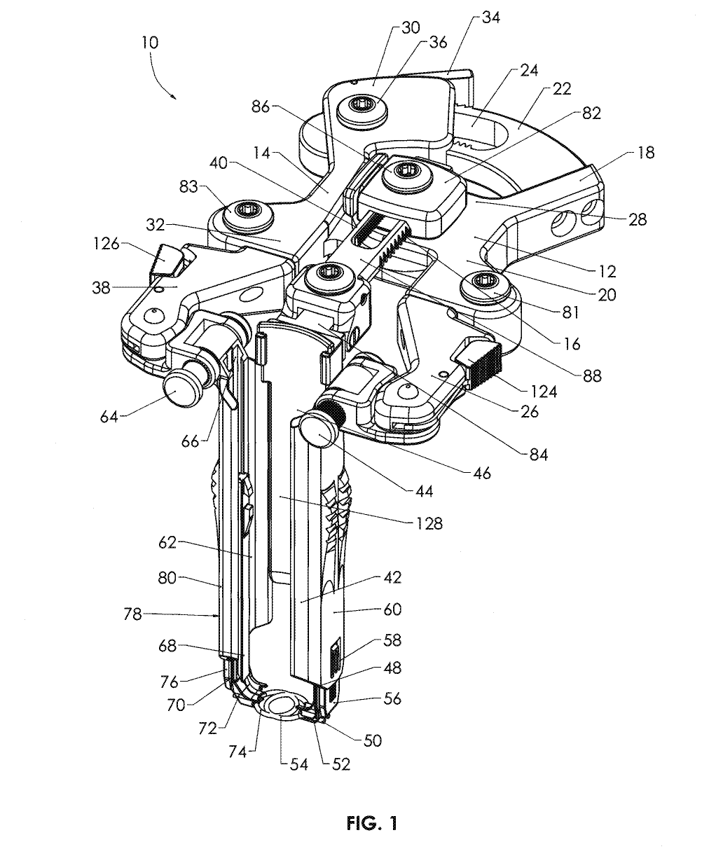

FIG. 2 is a perspective view of the retractor/distractor in an open position;

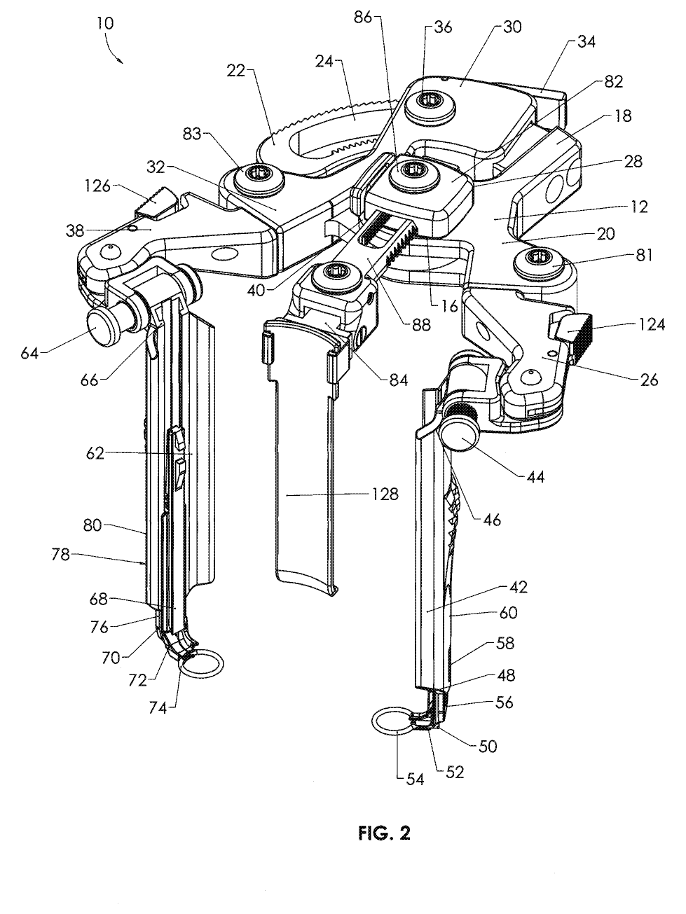

FIG. 3 is a top view of the retractor/distractor apparatus in a closed position;

FIG. 4 is a top view of the retractor/distractor apparatus in an open position;

FIG. 5 is a front exploded view of the retractor/distractor apparatus;

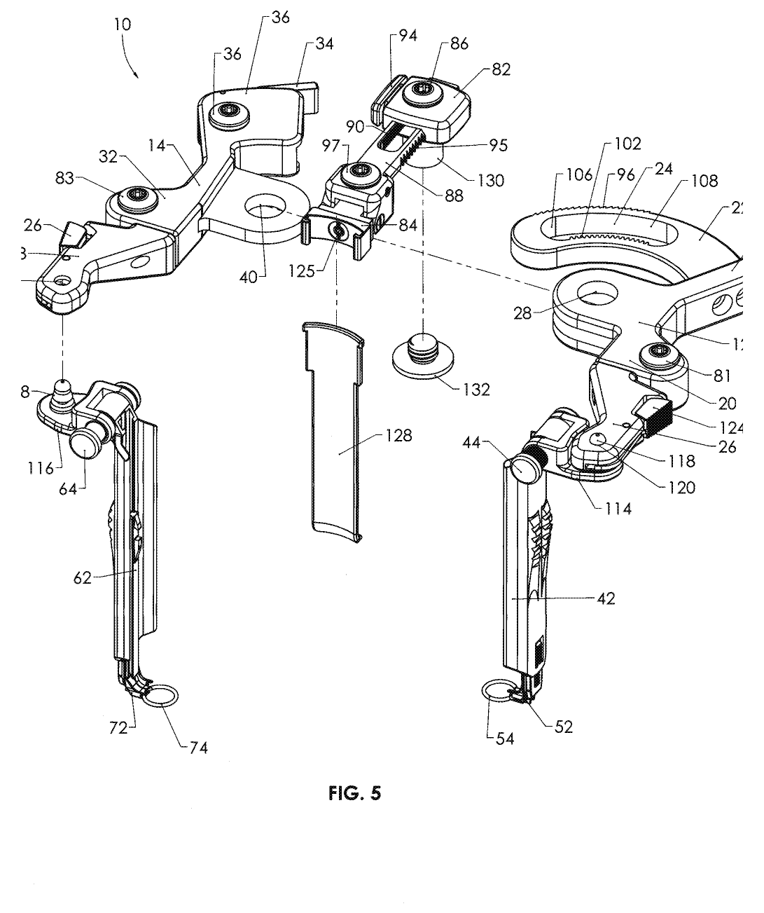

FIG. 6 is a rear exploded view of the retractor/distractor apparatus;

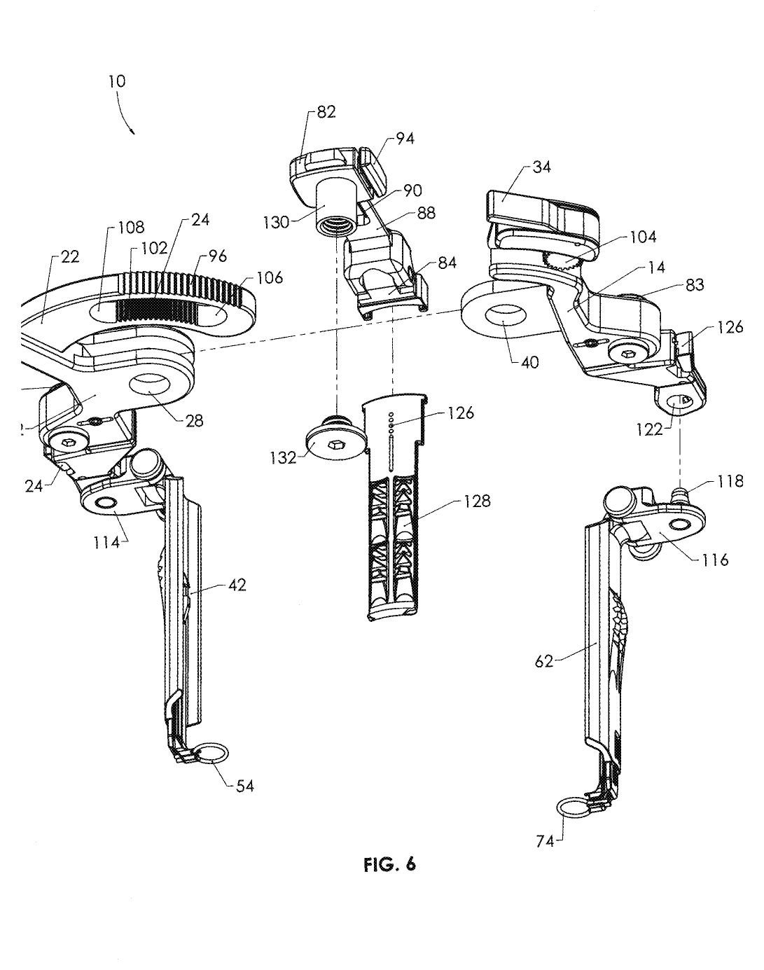

FIG. 7 is an exploded view of a blade assembly including a bone screw;

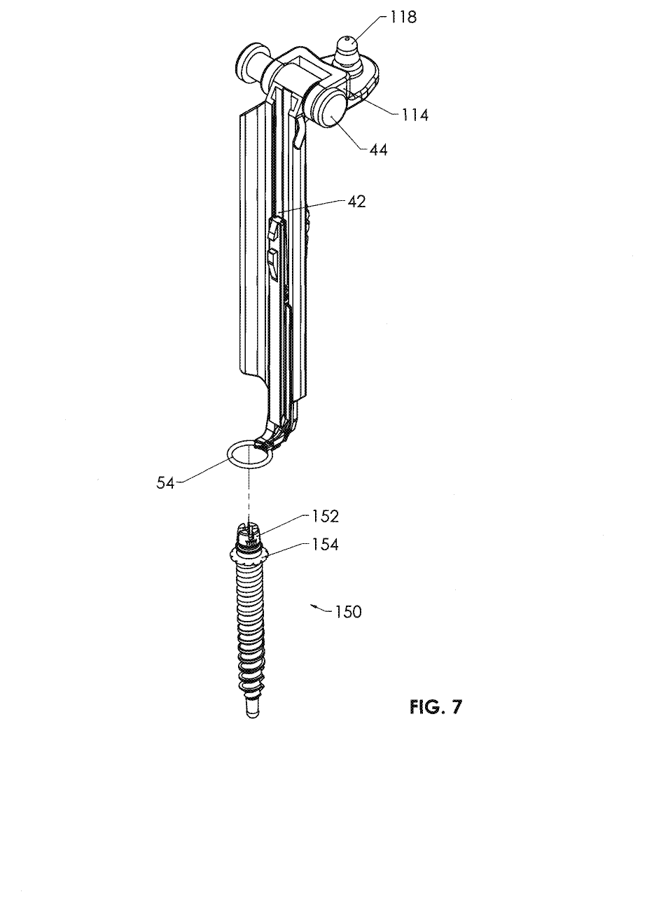

FIG. 8 is a perspective view of thereof with the bone screw attached to a hoop;

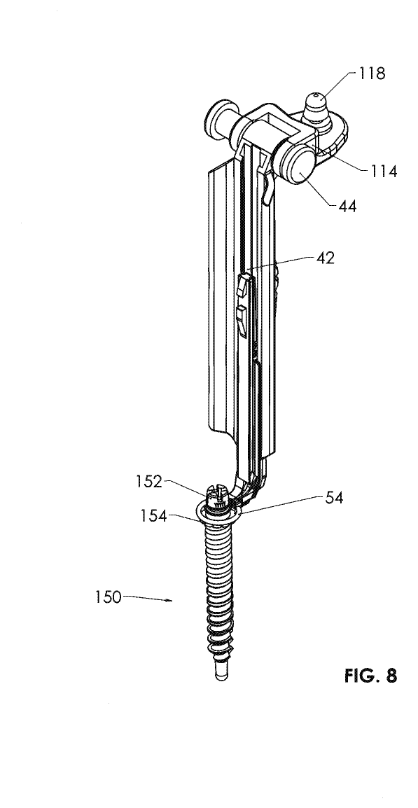

FIG. 9 is a cross-sectional view of the retractor/distractor apparatus along lines A-A; and

FIG. 10 is a cross-sectional view of the retractor/distractor apparatus along lines B-B.

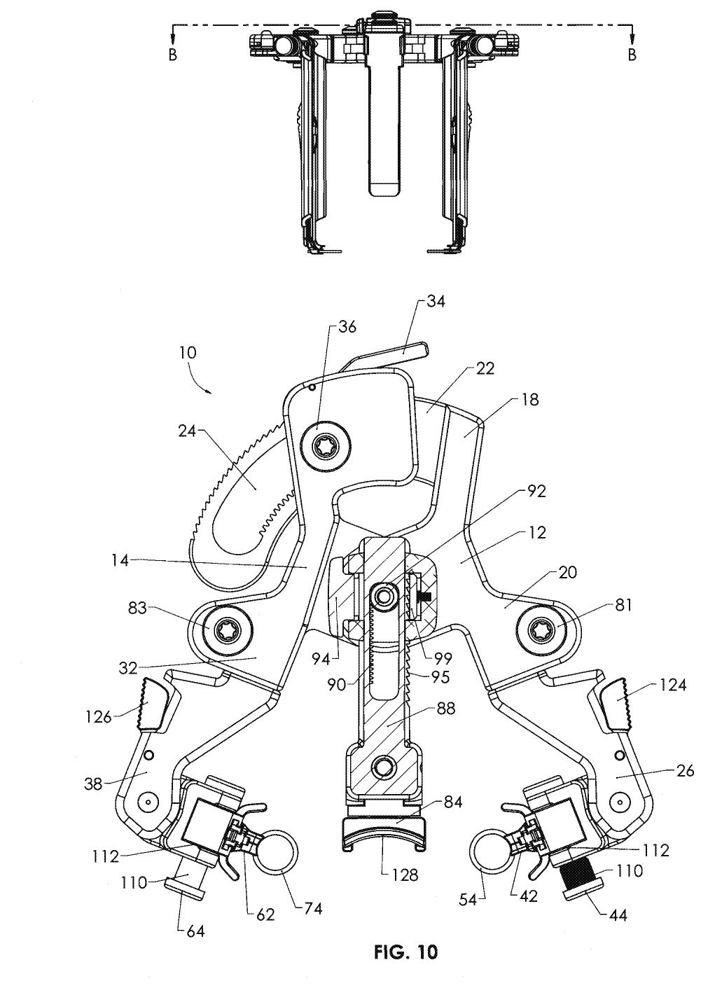

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

Referring now to the figures, illustrated is a retractor/distractor apparatus 10 constructed in accordance with the present invention. The apparatus 10 includes a first member 12 and a second member 14 coupled together at an offset pivot interface 16. The offset pivot interface 16 permits relative movement between the first member 12 and the second member 14. The first member 12 includes an upper leg 18 and a lower leg 20. The upper leg 18 supports a ratchet member 22 having a ratchet tooth drive 24, and the lower leg 20 is coupled to a first arm 26. The first member 12 further includes an offset pivot point 28 positioned at the offset pivot interface 16.

The second member 14 includes an upper end 30 and a lower end 32. The upper end 30 supports a ratchet lever 34 operatively associated with the ratchet tooth drive 24. A ratchet adjustment screw 36 is supported by the upper end 30 and is configured to actuate the ratchet lever 34. The lower end 32 is coupled to a second arm 38. The second member 14 further includes a reciprocal offset pivot point 40 that is pivotedly securable to the offset pivot point 28 of the first member 12 at the pivot interface 16. In this configuration, rotation of the ratchet adjustment screw 36 causes the second member 14 to move relative to the first member 12 along the ratchet member 22, thereby adjusting spacing between the first arm 26 and the second arm 38.

The first arm 26 and the second arm 38 are configured to support respective blade assemblies. A first blade 42 is coupled to the first arm 26 by a first attachment pin 44. The first blade 42 includes an upper blade opening 46 that receives the first attachment pin 44. The first blade 42 further defines a pathway 48 extending along opposing sides of the blade between the upper blade opening 46 and a lower end aperture 50. A first wire 52 extends through the pathway 48 and forms a hoop 54 at the lower end aperture 50. The hoop 54 is selectively expandable to releasably secure a bone anchor 150 during use. The bone anchor 150 may include a bone screw having a head portion 152 with a rib 154 configured to be received within the hoop 54. In this manner, the hoop 54 engages the rib 154 to secure the first blade 42 to the bone anchor 150. As used herein, the term “bone anchor” includes bone screws and other fixation elements configured to be secured within bone.

In certain embodiments, the first blade 42 includes a first adjustable extension portion 56 configured to vary a position of the lower end aperture 50 relative to the first arm 26. The first adjustable extension portion 56 permits the pathway 48 and the hoop 54 formed by the first wire 52 to be repositioned along a length of the first blade 42. In this manner, the location at which the hoop 54 engages a bone anchor 150 can be adjusted depending on the surgical requirements. The first adjustable extension portion 56 may further include a locking tab 58 disposed along a rear surface 60 of the first blade 42. The locking tab 58 is configured to selectively secure the first adjustable extension portion 56 in a desired position. The locking tab 58 may be actuated to permit movement of the first adjustable extension portion 56 and released to retain the first adjustable extension portion 56 in place.

The second arm 38 supports a second blade 62. The second blade 62 is coupled to the second arm 38 by a second attachment pin 64. The second blade 62 includes an upper blade opening 66 that receives the second attachment pin 64. The second blade 62 further defines a pathway 68 extending along opposing sides of the blade between the upper blade opening 66 and a lower end aperture 70. A second wire 72 extends through the pathway 68 and forms a hoop 74 at the lower end aperture 70. The hoop 74 is selectively expandable to releasably secure a bone anchor 150 during use. The bone anchor 150 may include a bone screw having a head portion 152 with a rib 154 configured to be received within the hoop 74. In this manner, the hoop 74 engages the rib 154 to secure the second blade 62 to the bone anchor 150.

In certain embodiments, the second blade 62 includes a second adjustable extension portion 76 configured to vary a position of the lower end aperture 70 relative to the second arm 38. The second adjustable extension portion 76 permits the pathway 68 and the hoop 74 to be repositioned along a length of the second blade 62. The second adjustable extension portion 76 may include a locking tab 78 disposed along a rear surface 80 of the second blade 62.

The first arm 26 is coupled to the lower leg 20 of the first member 12 by a first worm gear 81, and the second arm 38 is coupled to the lower end 32 of the second member 14 by a second worm gear 83. Each of the worm gears 81, 83 includes a threaded worm 85 configured to engage a corresponding gear portion 87 associated with the respective arm. Rotation of the worm gears 81, 83 causes controlled angular movement of the first arm 26 and the second arm 38, which in turn adjusts the angular orientation of the blade assemblies supported by the arms. The worm gears 81, 83 allow for fine angular adjustment of the arms and provide resistance to back-driving to maintain a selected position during use.

In certain configurations, a distal adjustment screw 97 is positioned at an end of the rack member 88 and is operatively associated with the medial retractor arm 84. Rotation of the distal adjustment screw 97 causes angular movement of the medial retractor arm 84 relative to the rack member 88. In this manner, the orientation of a medial retractor blade 128 supported by the medial retractor arm 84 can be adjusted independently of the translational position of the rack member 88.

The housing 82 is coupled to the offset pivot interface 16 and supports an adjustable medial retractor arm 84. The housing 82 includes a medial actuation screw 86 rotatably coupled to a rack member 88. The rack member 88 is configured to translate relative to the housing 82 in response to rotation of the medial actuation screw 86. In certain embodiments, the rack member 88 includes a plurality of inner teeth 90 that engage a pinion 92 supported by the housing 82. Rotation of the medial actuation screw 86 drives the pinion 92, which in turn advances or retracts the rack member 88 to position the medial retractor arm 84.

The housing 82 may further include a medial locking actuator 94 configured to selectively control movement of the medial retractor arm 84. The medial locking actuator 94 is operatively associated with a locking pawl 99 that is movable relative to the housing 82. The locking pawl 99 is configured to engage a plurality of rack outer teeth 95 formed on the rack member 88. Engagement between the locking pawl 99 and the rack outer teeth 95 prevents translation of the medial retractor arm 84 in a forward direction. Actuation of the medial locking actuator 94 causes the locking pawl 99 to disengage from the rack outer teeth 95, thereby permitting translation of the medial retractor arm 84 in a backward direction. In this manner, the medial retractor arm 84 can be incrementally advanced and selectively released to adjust positioning within the surgical site.

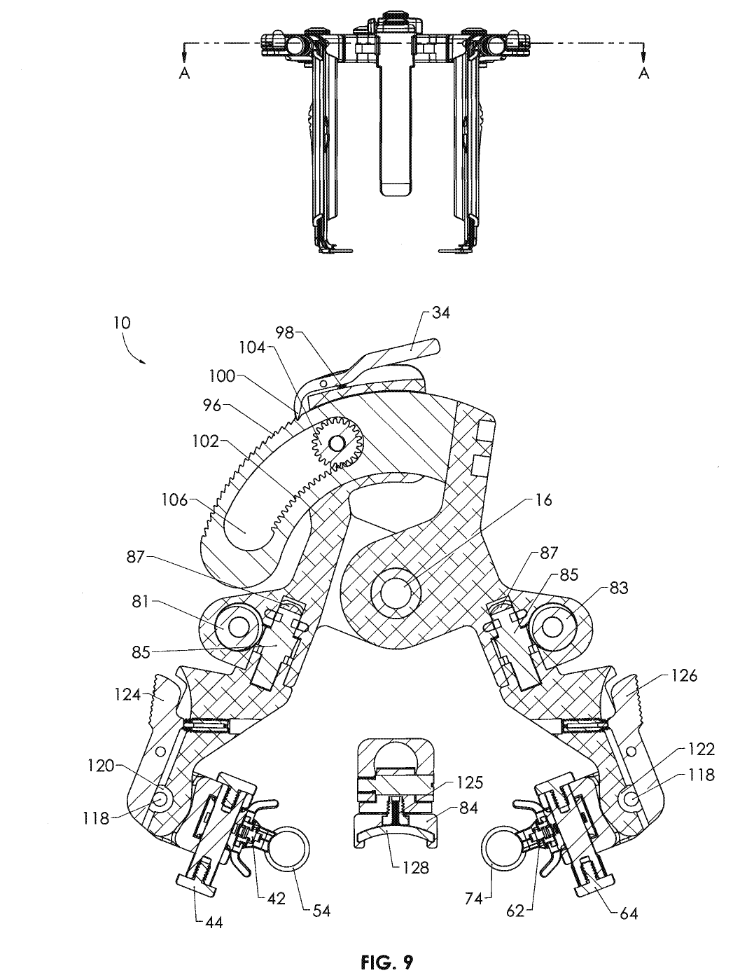

The ratchet member 22 extends from the upper leg 18 of the first member 12 and, in certain embodiments, is arcuate in shape. The ratchet member 22 includes the ratchet tooth drive 24 having a plurality of outer locking teeth 96 arranged along the arcuate profile. The ratchet lever 34 is positioned to engage the outer locking teeth 96. A click spring 98, shown in FIG. 9, is operatively associated with the ratchet lever 34 and biases an end 100 of the ratchet lever 34 into engagement with the outer locking teeth 96. In this configuration, rotation of the ratchet adjustment screw 36 advances the second member 14 relative to the first member 12 along the ratchet member 22 in a first direction. The engagement between the ratchet lever 34 and the outer locking teeth 96 resists movement in the opposite direction.

The ratchet lever 34 may be actuated to disengage from the outer locking teeth 96. Actuation of the ratchet lever 34 compresses the click spring 98 and moves the end 100 of the ratchet lever 34 away from the outer locking teeth 96. This allows the second member 14 to move relative to the first member 12 in the opposite direction along the ratchet member 22. In this manner, the spacing between the first arm 26 and the second arm 38 can be adjusted between a closed position and an open position.

In certain embodiments, the ratchet tooth drive 24 further includes a plurality of inner teeth 102 arranged along the ratchet member 22. The inner teeth 102 are distinct from the outer locking teeth 96 and are configured to engage a spread pinion 104 associated with the ratchet adjustment screw 36. Rotation of the ratchet adjustment screw 36 drives the spread pinion 104 along the inner teeth 102, thereby advancing or retracting the second member 14 relative to the first member 12 along the ratchet member 22.

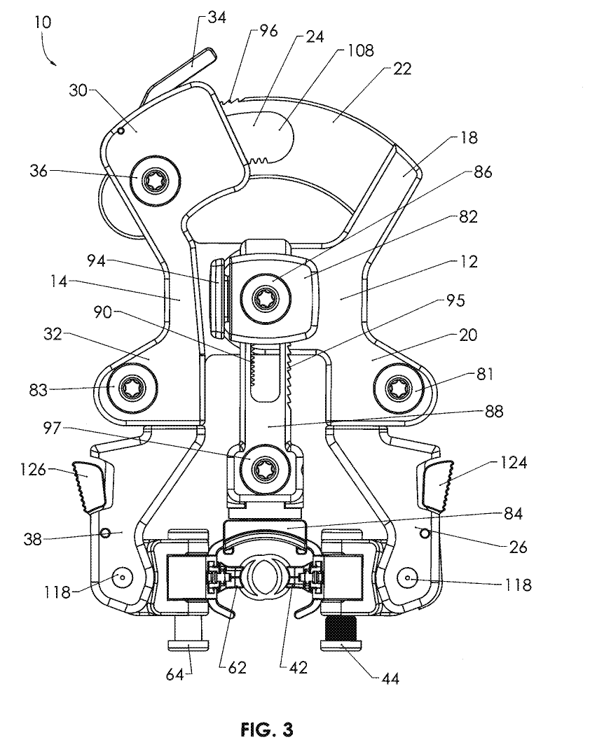

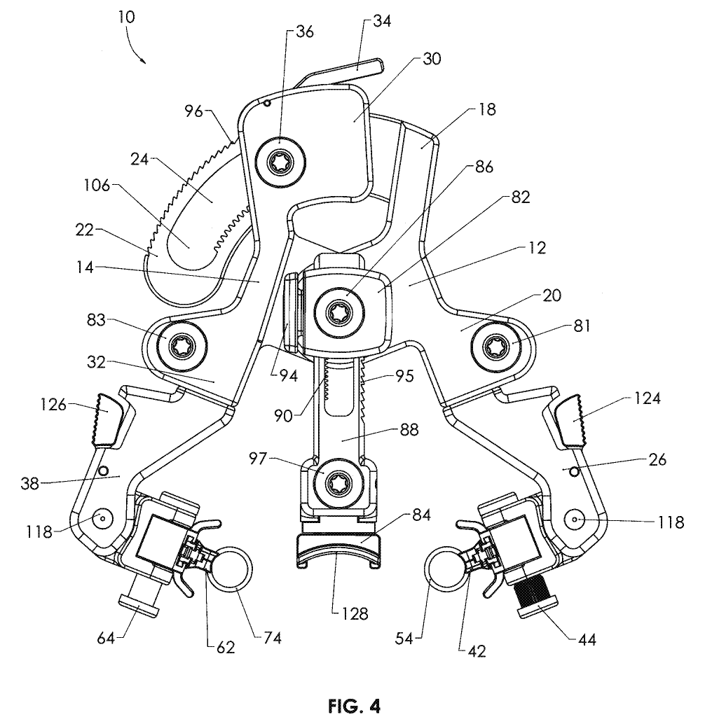

The apparatus 10 is shown in a closed position in FIG. 3, in which the first arm 26 and the second arm 38 are positioned closest to one another. In this position, the spread pinion 104 associated with the ratchet adjustment screw 36 is positioned toward a first end 106 of the ratchet member 22. In FIG. 4, the apparatus 10 is shown in an open position, in which the first arm 26 and the second arm 38 are spaced apart from one another. In this position, the spread pinion 104 is positioned toward an opposite end 108 of the ratchet member 22. Movement of the spread pinion 104 along the ratchet member 22 corresponds to controlled adjustment of spacing between the first arm 26 and the second arm 38.

In certain embodiments, the first attachment pin 44 and the second attachment pin 64 include splined surfaces 110 configured to engage corresponding splined openings 112 of the first blade 42 and the second blade 62, respectively. The splined surfaces 110 permit indexed angular positioning of the blades relative to the first arm 26 and the second arm 38. In this manner, the orientation of each blade can be adjusted and retained in a selected position.

In certain embodiments, the first blade 42 is coupled to a first blade bracket 114 and the second blade 62 is coupled to a second blade bracket 116. Each of the first blade bracket 114 and the second blade bracket 116 includes a catch post 118 configured to be received within an aperture 120 of the first arm 26 and an aperture 122 of the second arm 38, respectively. The catch post 118 may include a tapered or bulbous profile to facilitate insertion into the apertures 120, 122. The profile of the catch post 118 is not limiting. A first release button 124 may be provided on the first arm 26 and a second release button 126 may be provided on the second arm 38 to disengage the catch post 118 from the respective apertures 120, 122, thereby permitting removal of the blade assemblies from the arms.

In certain embodiments, the medial retractor arm 84 includes a detent 125 configured to engage a plurality of corresponding recesses 126 on a medial retractor blade 128. The detent 125 may include a spring-biased protrusion that selectively engages the recesses 126 to hold the medial retractor blade 128 in a desired position. The detent 125 permits incremental positioning of the medial retractor blade 128 and resists unintended movement during use. In certain embodiments, the detent 125 also facilitates removable attachment of the medial retractor blade 128 to the medial retractor arm 84, such that medial retractor blades of different sizes or configurations can be selectively installed.

In certain embodiments, a threaded barrel 130 extends through the offset pivot interface 16. The threaded barrel 130 passes through the offset pivot point 28 of the first member 12 and the reciprocal offset pivot point 40 of the second member 14. A retention screw 132 is threadably engaged with the threaded barrel 130 to secure the first member 12 and the second member 14 together. The retention screw 132 retains the first member 12 and the second member 14 in engagement with the threaded barrel 130 while permitting relative pivoting movement about the threaded barrel 130. The retention screw 132 may be configured to receive a driving tool, such as a hex key, a Torx® key, or another suitable driver. It should be understood that the configuration of the retention screw 132 is not limited to any particular drive type.

In use, the retractor/distractor apparatus 10 is positioned at a surgical site, such as during a spinal procedure, to provide controlled access to underlying anatomy. The blades 42, 62 may be secured to bone anchors 150 prior to attachment of the apparatus 10, allowing the surgeon to position the blades without obstruction. Once the apparatus 10 is coupled to the blades, the ratchet member 22 and ratchet adjustment screw 36 are used to adjust spacing between the first arm 26 and the second arm 38 to retract tissue. The medial retractor arm 84 may then be advanced or withdrawn using the medial actuation screw 86 to further adjust exposure. This allows the surgeon to position the blades and keep the surgical site open as needed during the procedure.

The term "coupled" is defined as connected, although not necessarily directly, and not necessarily mechanically. The use of the word "a" or "an" when used in conjunction with the term "comprising" in the claims and/or the specification may mean "one," but it is also consistent with the meaning of "one or more" or "at least one." The term "about" means, in general, the stated value plus or minus 5%. The use of the term "or" in the claims is used to mean "and/or" unless explicitly indicated to refer to alternatives only or the alternative are mutually exclusive, although the disclosure supports a definition that refers to only alternatives and "and/or."

The terms "comprise" (and any form of comprise, such as "comprises" and "comprising"), "have" (and any form of have, such as "has" and "having"), "include" (and any form of include, such as "includes" and "including") and "contain" (and any form of contain, such as "contains" and "containing") are open-ended linking verbs. As a result, a method or device that "comprises," "has," "includes" or "contains" one or more steps or elements, possesses those one or more steps or elements, but is not limited to possessing only those one or more elements. Likewise, a step of a method or an element of a device that "comprises," "has," "includes" or "contains" one or more features, possesses those one or more features, but is not limited to possessing only those one or more features. Furthermore, a device or structure that is configured in a certain way is configured in at least that way, but may also be configured in ways that are not listed.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments.

Indeed, various modifications of the described modes for carrying out the invention which are obvious to those skilled in the art are intended to be within the scope of the following claims.

What is claimed is:

1. . A rachet-based retractor and distractor apparatus comprising:
a first member having an upper leg and a lower leg, said upper leg coupled to a ratchet member having a ratchet tooth drive, said lower leg being coupled to a first arm, said first member further including an offset pivot point;
a second member having an upper end and a lower end, said upper end coupled to a ratchet lever operatively associated with said ratchet tooth drive and said lower end being coupled to a second arm, said second member further including a reciprocal offset pivot point pivotedly securable to said offset pivot point of said first member;
a first blade including a first attachment pin adapted to engage an upper blade opening at a proximal end of said first arm, said first blade defining a pathway extending between an upper blade opening and a lower end aperture along opposing sides of said blade;
a first wire having a first end extending through said pathway of said first blade and forming a hoop at said lower end aperture, said hoop being selectively expandable for releasably securing a bone screw;
a second blade including a second attachment pin adapted to engage an upper blade opening at a proximal end of said second arm, said second blade defining a pathway extending between an upper blade opening and a lower end aperture along opposing sides of said blade;
a second wire having a first end extending through the pathway of the second blade and forming a hoop at the lower end aperture, the hoop being selectively expandable to secure a bone screw releasably; and
a housing coupled to said offset pivot point for support of an adjustable medial retractor arm, said housing including a medial actuation screw rotatably coupled to a rack member wherein rotation of said medial actuation screw translates said medial retractor arm;
wherein said second member is operatively associated with said ratchet tooth drive such that rotation of a ratchet adjustment screw supported by said upper end of said second member causes said second member to move relative to said first member along said ratchet member to adjust spacing between said first arm and said second arm.

2. . The rachet-based retractor and distractor apparatus according to claim 1, wherein said ratchet tooth drive includes a plurality of outer locking teeth configured to permit movement of said second member in a first direction and resist movement in an opposite direction.

3. . The rachet-based retractor and distractor apparatus according to claim 2, wherein said ratchet lever includes an end configured to engage said plurality of outer locking teeth, and a click spring operatively associated with said ratchet lever to bias said end into engagement with said outer locking teeth, said click spring being compressible upon actuation of said ratchet lever to permit movement of said second member relative to said first member in the opposite direction.

4. . The rachet-based retractor and distractor apparatus according to claim 1, wherein said ratchet member is arcuate.

5. . The rachet-based retractor and distractor apparatus according to claim 1, wherein said rack member includes a plurality of inner teeth engaged with a pinion rotatably supported by said housing.

6. . The rachet-based retractor and distractor apparatus according to claim 1, wherein said housing further includes a medial locking actuator configured to selectively prevent translation of said medial retractor arm in a forward direction, said medial locking actuator being actuatable to permit translation of said medial retractor arm in a backward direction.

7. . The rachet-based retractor and distractor apparatus according to claim 1, wherein said first arm is coupled to said lower leg of said first member by a first worm gear and said second arm is coupled to said lower end of said second member by a second worm gear, wherein actuation of said first and second worm gears adjusts angular orientation of said first and second blades.

8. . The rachet-based retractor and distractor apparatus according to claim 1, wherein said first attachment pin and said second attachment pin include splined surfaces configured to engage corresponding splined openings of said first and second blades.

9. . The rachet-based retractor and distractor apparatus according to claim 1, wherein said first blade is coupled to a first blade bracket and said second blade is coupled to a second blade bracket, wherein said first blade bracket and said second blade bracket each include a catch post configured to be received within an aperture of said first arm and said second arm, respectively.

10. . The rachet-based retractor and distractor apparatus according to claim 1, wherein said medial retractor arm includes a detent configured to engage a plurality of corresponding recesses on said medial retractor blade.

11. . The rachet-based retractor and distractor apparatus according to claim 10, wherein said detent includes a spring-biased protrusion configured to selectively engage said recesses.

12. . A rachet-based retractor and distractor apparatus comprising:
a first member having an upper leg and a lower leg, said upper leg coupled to a ratchet member having a ratchet tooth drive, said ratchet tooth drive including a plurality of outer locking teeth configured to permit movement of a second member in a first direction and resist movement in an opposite direction, said lower leg being coupled to a first arm, said first member further including an offset pivot point;
a second member having an upper end and a lower end, said upper end coupled to a ratchet lever operatively associated with said ratchet tooth drive, said ratchet lever including an end configured to engage said plurality of outer locking teeth and a click spring operatively associated with said ratchet lever to bias said end into engagement with said outer locking teeth, said click spring being compressible upon actuation of said ratchet lever to permit movement of said second member relative to said first member in the opposite direction, and said lower end being coupled to a second arm, said second member further including a reciprocal offset pivot point pivotedly securable to said offset pivot point of said first member;
a first blade including a first attachment pin adapted to engage an upper blade opening at a proximal end of said first arm, said first blade defining a pathway extending between an upper blade opening and a lower end aperture along opposing sides of said blade;
a first wire having a first end extending through said pathway of said first blade and forming a hoop at said lower end aperture, said hoop being selectively expandable for releasably securing a bone screw;
a second blade including a second attachment pin adapted to engage an upper blade opening at a proximal end of said second arm, said second blade defining a pathway extending between an upper blade opening and a lower end aperture along opposing sides of said blade;
a second wire having a first end extending through the pathway of the second blade and forming a hoop at the lower end aperture, the hoop being selectively expandable to secure a bone screw releasably;
a housing coupled to said offset pivot point for support of an adjustable medial retractor arm, said housing including a medial actuation screw rotatably coupled to a rack member, said rack member including a plurality of inner teeth engaged with a pinion rotatably supported by said housing, wherein rotation of said medial actuation screw translates said medial retractor arm;
wherein said second member is operatively associated with said ratchet tooth drive such that rotation of a ratchet adjustment screw supported by said upper end of said second member causes said second member to move relative to said first member along said ratchet member to adjust spacing between said first arm and said second arm.

13. . The rachet-based retractor and distractor apparatus according to claim 12, wherein said ratchet member is arcuate.

14. . The rachet-based retractor and distractor apparatus according to claim 12, wherein said housing further includes a medial locking actuator configured to selectively prevent translation of said medial retractor arm in a forward direction, said medial locking actuator being actuatable to permit translation of said medial retractor arm in a backward direction.

15. . The rachet-based retractor and distractor apparatus according to claim 12, wherein said first arm is coupled to said lower leg of said first member by a first worm gear and said second arm is coupled to said lower end of said second member by a second worm gear, wherein actuation of said first and second worm gears adjusts angular orientation of said first and second blades.

16. . The rachet-based retractor and distractor apparatus according to claim 12, wherein said first attachment pin and said second attachment pin include splined surfaces configured to engage corresponding splined openings of said first and second blades.

17. . The rachet-based retractor and distractor apparatus according to claim 12, wherein said first blade is coupled to a first blade bracket and said second blade is coupled to a second blade bracket, wherein said first blade bracket and said second blade bracket each include a catch post configured to be received within an aperture of said first arm and said second arm, respectively.

18. . The rachet-based retractor and distractor apparatus according to claim 12, wherein said medial retractor arm includes a detent configured to engage a plurality of corresponding recesses on a medial retractor blade.

19. . The rachet-based retractor and distractor apparatus according to claim 18, wherein said detent includes a spring-biased protrusion configured to selectively engage said recesses.
